(19) United States
(12) Patent Application Publication
(10) Pub. No.: US 2006/0063927 A1
(43) Pub. Date: Mar. 23, 2006

(54) Processes for preparing quetiapine and salts thereof

(76) Inventors: Olga Etlin, Beer-Sheva (IL); Michael Brand, RaAnana (IL); Julia Ditkovich, Beer-Sheba (IL); Guy Davidi, Even-Yehuda (IL); Moty Shookrun, Petach-Tikva (IL); Oded Arad, Rehovot (IL); Joseph Kaspi, Givatayim (IL)

Correspondence Address: Martin Moynihan;PRTSI, Inc., P.O. Box 16446, Arlington, VA, 22215, US

(21) Appl. No.: 11/229,678

(22) Filed: Sep. 20, 2005

Related U.S. Application Data
(60) Provisional application No. 60/611,696, filed on Sep. 22, 2004.
(60) Provisional application No. 60/611,697, filed on Sep. 22, 2004.
(60) Provisional application No. 60/611,698, filed on Sep. 22, 2004.

(51) Int. Cl. C07D 498/02

(52) U.S. Cl. 540/551

(57) ABSTRACT

The present invention provides herein a two-step process for preparing pharmaceutically pure quetiapine and salts thereof by obtaining the starting material 11-chloro-dibenzo-thiazepine followed by reacting the 11-chloro-dibenzo-thiazepine with 1-(2-hydroxyethoxy)ethylpiperazine, or its salt, in the presence of an inorganic or organic base in an organic solvent or in a two-phase solvent system. The present invention provides also a novel, one-pot reaction process for preparing pharmaceutically pure quetiapine and salts thereof. The two processes provided herein can be easily, conveniently and inexpensively scaled-up.

### RELATED APPLICATIONS

The present application claims priority from U.S. Provisional Patent Applications Nos. 60/611,696, 60/611,697, and 60/611,698, all filed on Sep. 22, 2004, which are incorporated herein by reference.

### FIELD OF THE INVENTION

The present invention relates to improved processes for preparing quetiapine {11-(4-[2-(2-hydroxyethoxy)ethyl]-1-piperazinyl)dibenzo[b,f][1,4] thiazepine} and salts thereof.

### BACKGROUND OF THE INVENTION

11 -(4-[2-(2-hydroxyethoxy)ethyl]-1-piperazinyl)dibenzo[b,f][1,4]thiazepine fumarate (1), also known as quetiapine fumarate, is a novel dibenzothiazepine antipsychotic drug, useful for treating schizophrenia, having the following molecular structure:

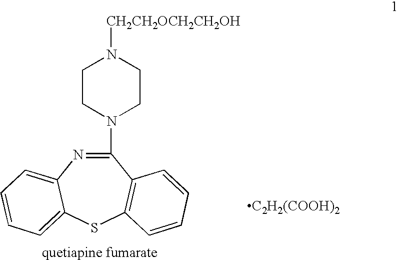

Quetiapine fumarate was developed by Zeneca and is marketed under the trade name Seroquel®.

Quetiapine and its preparation are disclosed in U.S. Pat. No. 4,879,288 (to Warawa et al). More specifically, this patent discloses a process for preparing quetiapine by converting dibenzo[b,f][1,4]thiazepine11(10-H)-one, compound 2, to 11-chloro-dibenzo[b,f][1,4]-thiazepine, compound 3 (hereinafter the imino chloride), by using 15 fold excess of phosphorous oxichloride, thus the chlorinating agent serves also as a dehydrating agent, followed by treating the thus obtained 11 -chloro-dibenzo[b,f][1,4]-thiazepine dissolved in xylene with an excess of 1-(2-hydroxyethoxy)ethylpiperazine, compound 4, to yield the crude quetiapine free base as a viscous amber-colored oil, which is converted to quetiapine hemi-fumarate.

The process for obtaining quetiapine hemi-fumarate, as taught in U.S. Pat. No. 4,879,288, is depicted in scheme 1.

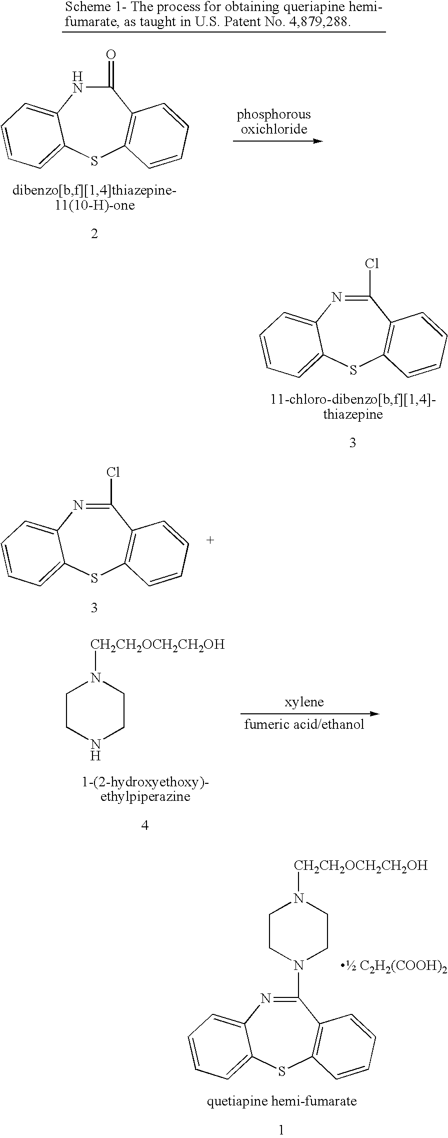

The oily crude product is purified by column chromatography to yield pure quetiapine free base in 77.7% yield. The free base product is converted to its hemi-fumarate salt by reacting it with fumaric acid in ethanol.

This process of preparing quetiapine teaches the use of column chromatography as an essential purification step for obtaining quetiapine as a pure free base. Column chromatography is a complicated, expensive, and inconvenient unit operation, which uses in some cases harmful or toxic solvents and therefore it is an environmentally unfriendly technique, which cannot be advantageously used for industrial large-scale production.

U.S. Pat. No. 4,879,288 discloses a second process for preparing quetiapine hemi-fumarate, by reacting compound 3 with piperazine in warm toluene to obtain 11-piperazinyl-dibenzo[b,f][1,4]-thiazepine, compound 5, as the dihydrochloride salt. Compound 5 is then reacted with 2-chloroethoxyethanol in the presence of sodium carbonate and a catalytic amount of sodium iodide in a mixture of n-propanol or toluene with N-methylpyrrolidone to obtain quetiapine as an oily free base. The oily free base product is converted to its hemi-fumarate salt by reacting it with fumaric acid in ethanol.

The second process for obtaining quetiapine, as taught in U.S. Pat. No. 4,879,288, is depicted in Scheme 2.

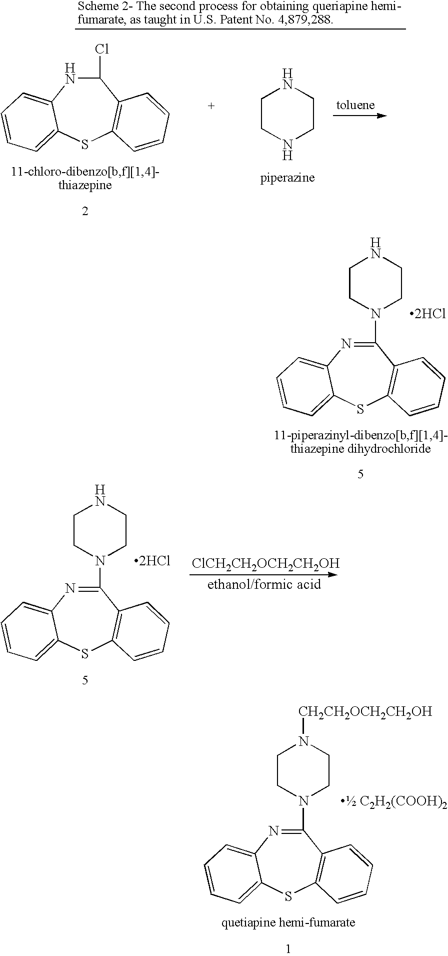

Other processes for preparing quetiapine are known in the literature for example the process developed by Egis Gyogyszergyar Rt., which is provided in European Patent No. 1252151. The process comprises treating phenyl N-[2-(phenylthio)phenyl]carbamate, compound 6, with 1-(2-hydroxyethyl)piperazine, simultaneous reaction of the resulting hydroxyethylpiperazine derivative, compound 7, with a halogenating agent (such as phosphorus oxychloride), which is used in a 3 fold excess thus the chlorinating agent serves also as a dehydrating agent, to obtain a haloethylpiperazinylthiazepine, and conversion to quetiapine by reaction with ethylene glycol.

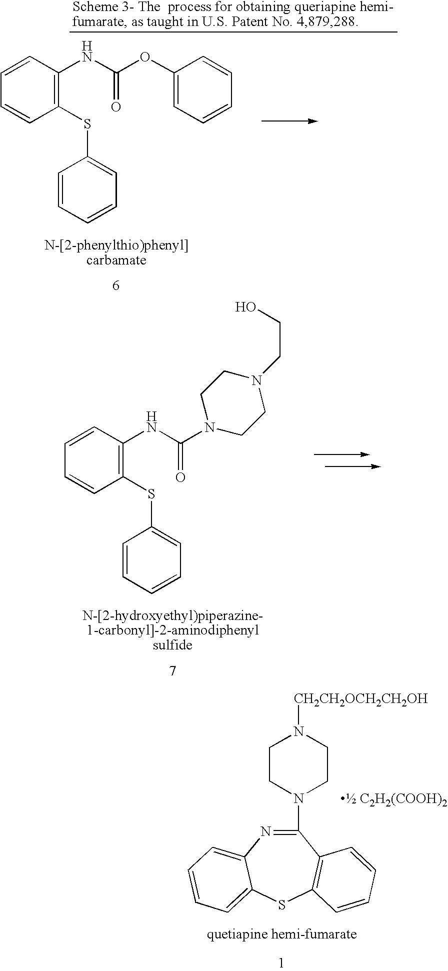

The object of the present invention is to provide efficient, economic and environmentally friendly processes for preparing quetiapine, avoiding using column chromatography; hence the use of organic solvents is minimized. These processes may be easily, conveniently and inexpensively scaled-up for industrial large-scale production.

### SUMMARY OF THE INVENTION

The present invention provides improved processes for preparing quetiapine and salts thereof in a two-step reaction as well as in a one-pot reaction.

In one embodiment, the present invention provides low cost and environmentally friendly processes for preparing pure quetiapine and salts thereof by minimizing the use of harmful organic reagents and solvents.

In another embodiment of the present invention, an alternative process to one taught in U.S. Pat. No. 4,879,288, which is depicted in scheme 1, is provided for preparing quetiapine, wherein the first step comprises obtaining the imino chloride avoiding using phosphorus oxychloride, phosphorus pentachloride or thionyl chloride. More specifically, the object of the present invention is to provide an alternative process for preparing quetiapine, wherein the first step of the process comprises reacting 10H-dibenzo[b,f][1,4]thiazepine-11-one, compound 2, with oxalyl chloride to yield the desired product.

In one aspect, the present invention provides a process for preparing quetiapine, wherein the first step comprises obtaining the imino chloride.

In another aspect, the present invention provides a process for preparing quetiapine, wherein the first step comprises obtaining the imino chloride while avoiding the use of phosphorus oxychloride, phosphorus pentachloride or thionyl chloride.

According to the present invention, the imino chloride is prepared by reacting dibenzo[b,f][1,4]thiazepine-11(10-H)-one, compound 2, with oxalyl chloride in an organic solvent to yield imino chloride, compound 3.

In one embodiment, the present invention provides an efficient, economic and also environmentally friendly process for preparing quetiapine and salts thereof, wherein the second step of the process comprises reacting 11-chloro-dibenzo[b,f][1,4]-thiazepine, compound 3, with 1-(2-hydroxyethoxy)ethylpiperazine, compound 4, or its salt, in the presence of an inorganic or organic base in an organic solvent or in a two-phase solvent system.

The two-phase solvent system, in the context of the present invention, is prepared by combining a water immiscible organic solvent in which compounds 3 and compound 4 are soluble in and an aqueous phase in which the inorganic base is soluble in to form a solvent system comprising two phases. Preferred organic solvents are aromatic organic solvents and more preferred are aromatic organic solvents selected from the group consisting of ethylbenzene, toluene and xylenes.

In another aspect of the present invention, once the reaction is complete, quetiapine can be conveniently separated from impurities such as unreacted starting material, organic and inorganic salts and side-products by conventional physical separation (such as filtration, extraction, etc.). In addition to the processes themselves, the present invention further provides quetiapine and pharmaceutically acceptable salts thereof prepared by conventional methods known in the art.

In another aspect, the present invention provides a novel one-pot process for preparing pure quetiapine and salts thereof.

In another aspect, the present invention provides a one-pot process for preparing pure quetiapine and salts thereof that offers an advantage to industrial processes since complicated separation and purification steps can be avoided and the expenditure on equipment can be reduced.

In yet another aspect, the present invention provides a one-pot process for preparing pure quetiapine and salts thereof comprising reacting dibenzo[b,f][1,4]thiazepine-11(10-H)-one, compound 2, with halogenating agent in a solvent, to yield compound 3 in situ and reacting the thus obtained product with 1-(2-hydroxyethoxy)ethylpiperazine, compound 4, or its salt in the presence of a free base.

In another aspect of the present invention, once the reaction is complete, quetiapine can be conveniently separated from impurities such as unreacted starting material, organic and inorganic salts and side-products by conventional physical separation (such as filtration and extraction) of the impurities from the reaction mixture.

In addition to the processes themselves, the present invention further provides quetiapine and pharmaceutically acceptable salts thereof prepared by conventional methods known in the art.

Pure quetiapine, in the context of the present invention refers to a product containing quetiapine free base or a pharmaceutically acceptable salt thereof and impurities in an amount of less than about 2% w/w and preferably, less than about 1% w/w, more preferably less than about 0.5% w/w and most preferably less than about 0.1% w/w, in respect to the total weight of the product.

### DESCRIPTION OF THE PREFERRED EMBODIMENTS

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The present invention meets a need in the art for improved, low cost and environmentally friendly processes for preparing quetiapine and acceptable pharmaceutical salts thereof, avoiding using column chromatography.

In all of the presently known processes of preparing quetiapine, chlorinating agents such as phosphorus oxychloride, thionyl chloride and phosphorus pentachloride, being some of the most readily available chlorinating agents around the world, are utilized. These chlorinating agents are considered hazardous and in some countries, shipping into and/or ground transportation thereof is restricted.

An alternative process of preparing quetiapine, which circumvents the need to use such environmentally unfriendly reagents, is advantageous. Furthermore, contrary to the teaching of the processes described in U.S. Pat. No. 4,879,288 and European Patent No. 1252151, the processes of the present invention use equimolar amount or slight excess of a chlorinating agent such as oxalyl chloride, while quetiapine can be readily obtained thereof in relatively high yield and purity. Using a large excess of a chlorinating agent such as phosphorus oxychloride is disadvantageous because of the environmental problems which are concerned with disposal of solvent waste containing high concentration of acid.

The present invention provides improved processes for preparing quetiapine and salts thereof in a two-step reaction as well as in a one-pot reaction, as depicted in scheme 4, wherein dibenzo[b,f][1,4]thiazepine-11(10-H)-one is chlorinated with equimolar or slight excess of oxalyl chloride, thus the present invention meets a need in the art for an alternative process for preparing the imino chloride that avoids using phosphorus oxychloride, phosphorus pentachloride or thionyl chloride.

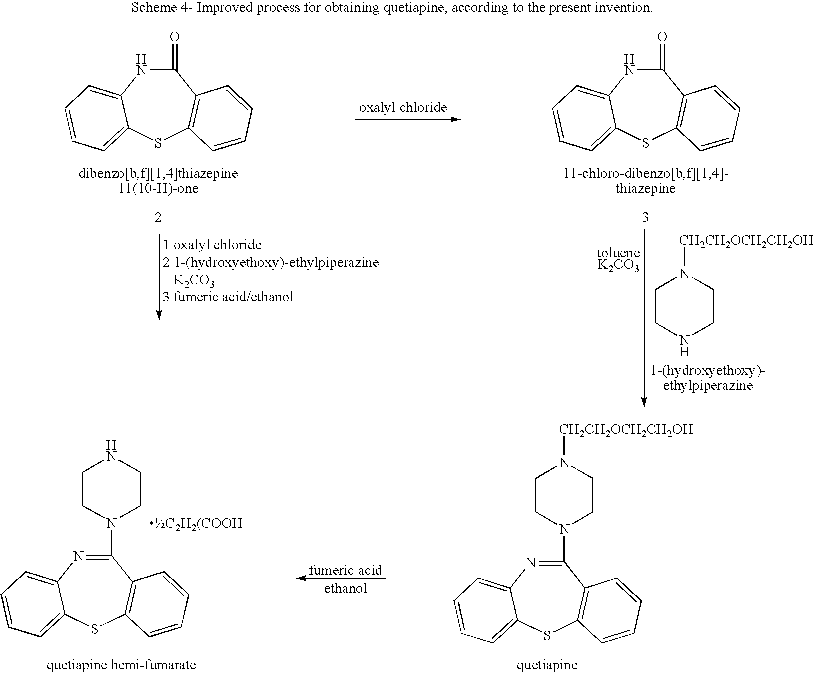

Dibenzo[b,f][1,4]thiazepine-11(10-H)-one, compound 2, used as the starting material in the embodiments disclosed hereinafter, is a known compound and obtainable, e.g., by conventional methods known in the art.

In accordance with the present invention there is provided a process for preparing quetiapine and the corresponding acid addition salts thereof, comprising the steps of:
a) Reacting dibenzo[b,f][1,4]thiazepine-11(10-H)-one, compound 2, with halogenating agent in an organic solvent to yield compound 3; and
b) Reacting the product resulting from step a) with 1-(2-hydroxyethoxy)ethylpiperazine, compound 4, or its salt, in the presence of a base.

In accordance with the present invention the first step a) in the process for obtaining quetiapine and the corresponding acid addition salts thereof comprises:

a) Charging the reaction vessel with an organic solvent and optionally drying the organic solvent by azeotropic distillation;

b) Adding an amide and optionally cooling the reaction mixture;

c) Adding a chlorinating agent drop-wise followed by adding compound 2;

d) Refluxing the reaction mixture for sufficient time to allow completing the reaction; and

e) Working-up the reaction mixture thus obtaining compound 3.

In one embodiment of the present invention, the process is carried out in an organic solvent.

In another embodiment of the present invention, the organic solvent may be used as is or may be dried prior to use by any conventional method known in the art.

In another embodiment of the present invention the organic solvent may be maintained water-free during the process by continuous removal of water or, for example, using a Dean-Stark trap or activated molecular sieves.

As used herein, the term “organic solvent” means a solvent conventionally understood as such in the art, including a solvent in which non-polar or hydrophobic compounds are preferentially and substantially soluble.

In a preferred embodiment of the present invention, the process is carried out in a non-polar aprotic solvent.

In another preferred embodiment of the present invention, the process is carried out in an aromatic solvent. Non limiting examples of aromatic solvents usable in context of the present invention include ethylbenzene, toluene, xylenes, and the like and mixtures thereof.

In a more preferred embodiment of the present invention, the process is carried out in toluene.

Non-limiting examples of halogenating agents that can be used in the present invention include thionyl chloride, phosphorous pentachloride, phosphorous oxychloride, oxalyl chloride, N-chlorosuccinimide or N-chlorobenzotriazole. Preferably, the chlorinating agent is oxalyl chloride.

In another embodiment of the present invention, oxalyl chloride is added into a cooled reaction mixture, preferably the oxalyl chloride is added slowly, more preferably the oxalyl chloride is added drop-wise into the cooled reaction mixture. After the oxalyl chloride addition is complete the temperature is raised.

In another embodiment of the present invention, the oxalyl chloride is added to the reaction mixture at a temperature in the range of from −5° C. to 10° C., preferably of about 0° C.

In a further embodiment of the present invention, the process is carried out in the presence of an organic amide. Examples of such organic amides include cyclic and acyclic C₁ to C₆ amides such as N,N-dimethylacetamide (DMA), N-methylpyrrolidone (NMP) and N,N-dimethylformamide (DMF). In the context of the present invention N,N-dimethylformamide is preferred.

In another embodiment of the present invention, the process is conveniently conducted at ambient temperature or at an elevated temperature, preferably at a temperature between ambient and the reflux temperature of the reaction mixture, more preferably at the reflux temperature.

In another embodiment of the present invention, the reaction is carried out for an extended period of time, preferably from about 1 hour to about several days, more preferably from about 5 hours to about 24 hours and most preferably about 21 hours.

In a preferred embodiment of the present invention, the reaction mixture is evaporated after the imino chloride, compound 3, is prepared.

In a further embodiment of the present invention, the mixture is washed several times with water.

In yet another embodiment of the present invention, to assist in impurity removal, it is effective to treat the substrate with an adsorbent, preferably with active charcoal. Activated charcoal is added to the organic solution containing the imino chloride. If desired, a filter-aid may be additionally added. After the activated charcoal has been added, stirring is continued at constant temperature for between 5 and 60 minutes, preferably between 10 and 30 minutes, most preferably about 15 minutes, and the mixture obtained is filtered to remove the solids.

In yet another embodiment of the present invention, after the solids are removed, the mixture is evaporated to dryness.

After removing the impurities, the imino chloride product is recovered, for example by separation from the solvent or other volatiles. In a preferred embodiment, the pressure is reduced, and the temperature is raised slightly to evaporate the solvent or other volatiles. After evaporation, a brown-colored product is obtained in 66% yield. The purity of the product obtained by this process is preferably greater than 90% as measured by HPLC.

In accordance with the present invention, the second step b) in the process for preparing quetiapine and the corresponding acid addition salts thereof comprises:

a) Reacting 11-chloro-dibenzo[b,f][1,4]-thiazepine, compound 3, with 1-(2-hydroxyethoxy)ethylpiperazine or its salt, compound 4, in the presence of a base in an organic solvent or in a two-phase solvent system;

b) Filtering the reaction mixture, washing and optionally treating the organic phase with activated charcoal; and

c) Isolating the pure product as a corresponding acid addition salt thereof.

In another embodiment of the present invention, the reaction is carried out in the presence of a base in an organic solvent, while the base can be either an inorganic base or an organic base.

Non-limiting examples of the inorganic base can include metal hydroxides such as sodium hydroxide and potassium hydroxide; metal carbonates such as sodium carbonate and potassium carbonate; and metal hydrogen carbonates such as sodium hydrogen carbonate and potassium hydrogen carbonate.

In a preferred embodiment of the present invention, the inorganic base is selected from the group consisting of metal carbonates such as sodium carbonate and potassium carbonate, more preferred inorganic base in the context of the present invention is potassium carbonate.

Non-limiting examples of the organic base can include tertiary alkylamines such as triethylamine, tributylamine and N,N-diisopropylethylamine; dialkylanilines such as N,N-dimethylaniline and N,N-diethylaniline; heterocyclic amines such as pyridine, N,N-dimethylaminopyridine and N-methylmorpholine; metal alkoxides such as sodium methoxide, sodium ethoxide, sodium isopropoxide and potassium tert-butoxide; 1,8-diazobicyclo[5,4,0]undecene, N-benzyltrimethylammonium hydroxide, or a combination thereof.

In another preferred embodiment of the present invention, the organic base is selected from the group consisting of tertiary alkylamines such as triethylamine, tributylamine and N,N-diisopropylethylamine, whereby a more preferred organic base in the context of the present invention is triethylamine.

In another embodiment of the present invention, the amount of the base to be used may be in a range of from 0.1 to 15 molar ratio, preferably from 1 to 5 molar ratio or so relative to the molar number of 1-(2-hydroxyethoxy)ethylpiperazine, compound 4, or its salt.

In another embodiment of the present invention, 1-(2-hydroxyethoxy)-ethylpiperazine, compound 4, may be used as a free base or as the acid addition salt thereof.

In a preferred embodiment of the present invention, the reaction is carried out in the presence of an aromatic solvent. Non-limiting examples of aromatic solvents usable in context of the present invention include ethylbenzene, toluene, xylenes and the like, and mixtures thereof.

In a more preferred embodiment of the present invention, the reaction is carried out in the presence of toluene.

In yet another embodiment of the present invention, the reaction is carried out in a two-phase solvent system.

In another preferred embodiment of the present invention, the two-phase solvent system is prepared by mixing a water immiscible organic solvent, in which compounds 3 and 4 are soluble in, and an aqueous phase in which the inorganic base is soluble in to form a solvent system comprising two phases. Preferred water immiscible solvents are aromatic organic solvents and more preferred are aromatic organic solvents selected from the group consisting of ethylbenzene, toluene, xylenes, and the like and mixtures thereof.

In another embodiment of the present invention, while using the two-phase solvent system, the aqueous phase content is from about 1% w/w to about 60% w/w, preferably from about 10% w/w to about 50% w/w.

In another embodiment of the present invention, while using the two-phase solvent system, 1-(2-hydroxyethoxy)ethylpiperazine or its salt is added in a small excess, preferably the excess ranges from 0 to about 50% relative to 11-chloro-dibenzo[b,f][1,4]-thiazepine, more preferably from about 5% to about 25% and most preferably about 10%.

In another embodiment of the present invention, the reaction is conveniently conducted at an elevated temperature, preferably between 60° C. and reflux temperature, or at reflux temperature.

In another embodiment of the present invention, the reaction is carried out for an extended period of time, preferably from about 1 hour to about several days, more preferably from about 5 hours to about 60 hours.

In yet another embodiment of the present invention, the impurities may be optionally removed by conventional techniques well-known in the art. The impurities described hereinabove can be removed, if desired, by any suitable separation procedure such as, for example, filtration, extraction, column chromatography, thin-layer chromatography, preparative low, medium or high-pressure liquid chromatography or a combination of these procedures. Specific illustrations of suitable separation procedures are described in the Examples section that follows. However, other equivalent separation or isolation procedures could also be used. Filtration, extraction and a combination of these procedures are the presently most preferred separation procedures.

In yet another embodiment of the present invention, to assist in impurity removal, it is effective to treat the substrate with an adsorbent, preferably with active charcoal. Activated charcoal is added to the organic phase containing quetiapine free base. If desired, a filter-aid may be additionally added. After the activated charcoal has been added, stirring is continued at constant temperature for between 5 and 60 minutes, preferably between 10 and 30 minutes, most preferably about 15 minutes, and the mixture obtained is filtered to remove the solids.

In another embodiment of the present invention, the pure product may be converted to a corresponding acid addition salt. Preferably, these salts are pharmaceutically acceptable salts.

In yet another embodiment of the present invention, the conversion is accomplished by treatment with at least a stoichiometric amount of an appropriate acid. In the present invention, the appropriate acid includes, but is not limited to, inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. A preferred salt is the hemi-fumarate salt.

Typically, the quetiapine free base prepared by the process described hereinabove is dissolved in an alkanol solvent such as ethanol or methanol and the like, and the acid is added to the mixture. The temperature is maintained between 0° C. and the reflux temperature, preferably the temperature is maintained between 0° C. and 25° C. The resulting crystals precipitate spontaneously or may be brought out of solution by cooling the mixture or by adding a less polar solvent.

In yet another embodiment of the present invention, the isolated crystals can be dried using conventionally known methods to give pure quetiapine hemi-fumarate. The drying stage may be carried out by increasing the temperature or reducing the pressure or a combination of both. Non-limiting examples of drying technologies or equipments usable in context of the present invention include vacuum ovens, tray ovens, rotary ovens and fluidized bed dryers.

In another embodiment of the present invention, the crystalline quetiapine salt may be further purified by converting the acid addition salts to the corresponding free base by treatment with at least a stoichiometric equivalent of a suitable organic or inorganic base such as described hereinabove and converting the pure free base product again to a corresponding acid addition salt.

In another embodiment of the present invention, the crystalline quetiapine hemi-fumarate may be recrystallized by any conventional recrystallization method known in the art.

The above preparation process results in a pharmaceutically pure quetiapine hemi-fumarate, which may comprise impurities in an amount of less than about 3% w/w and preferably, less than about 2% w/w, more preferably less than about 1% w/w, more preferably less than about 0.5% w/w and most preferably less than about 0.1% w/w, in respect to the total weight of the product. As described in the examples, quetiapine hemi-fumarate can be obtained in a purity greater than 99% and preferably in a purity equal to or greater than 99.8%

The yield of the process is an important feature of the invention. As described in the examples, quetiapine hemi-fumarate can be obtained in yields of up to 92% with respect to the starting material.

In yet another embodiment of the present invention, the processes described hereinabove for preparing quetiapine and for the corresponding acid addition salts thereof may be conveniently and inexpensively scaled-up.

The present invention is predicated also on the unexpected finding that it is not necessary to carry out the reaction for obtaining quetiapine and the corresponding acid addition salts thereof in a two-step process and a one-pot process may be used instead.

In another embodiment of the present invention, the two-step reaction for obtaining quetiapine and the corresponding acid addition salts thereof, wherein step a) comprises chlorinating dibenzo[b,f][1,4]thiazepine-11(10-H)-one to 11-chloro-dibenzo[b,f][1,4]-thiazepine, and step b) comprises reacting 11-chloro-dibenzo[b,f][1,4]-thiazepine with 1-(2-hydroxyethoxy)ethylpiperazine, may be carried out in a single pot thereby avoiding the unit operation of isolation of the intermediate 11-chloro-dibenzo[b,f][1,4]-thiazepine.

In accordance with the present invention, an improved one-pot process for preparing quetiapine and the corresponding acid addition salts thereof is provided. The process comprises reacting dibenzo[b,f][1,4]thiazepine-11(10-H)-one, compound 2, with halogenating agent in an organic solvent to yield compound 3 in situ and reacting the thus obtained compound 3 with 1-(2-hydroxyethoxy)ethylpiperazine, compound 4, or its salt in the presence of a base.

In another embodiment of the present invention, the organic solvent may be used as is or may be dried prior to use by any conventional method known in the art.

In another embodiment of the present invention the organic solvent may be maintained water free during the reaction by continuous removal of water using, for example, a Dean-Stark trap or activated molecular sieves.

In a preferred embodiment of the present invention, the reaction is carried out in an aromatic solvent. Non-limiting examples of aromatic solvents usable in the context of the present invention include ethylbenzene, toluene, xylenes and the like and mixtures thereof.

In a more preferred embodiment of the present invention, the reaction is carried out in toluene.

In yet another embodiment of the present invention, dibenzo[b,f][1,4]thiazepine-11(10-H)-one, compound 2, is reacted with a halogenating agent, preferably a chlorinating agent. Non-limiting examples of chlorinating agents that can be used in the present invention include thionyl chloride, phosphorous pentachloride, phosphorous oxychloride, oxalyl chloride, N-chlorosuccinimide or N-chlorobenzotriazole. Preferably, the chlorinating agent is oxalyl chloride.

In a preferred embodiment of the present invention, oxalyl chloride is added into a cooled reaction mixture, preferably slowly, more preferably drop-wise. The reaction mixture is cooled during the oxalyl chloride addition. After the oxalyl chloride addition is complete the temperature is raised.

In yet another preferred embodiment of the present invention, oxalyl chloride is added to the reaction mixture at a temperature in the range of from −5° C. to 10° C, preferably of about 0° C.

In a further embodiment of the present invention, the reaction is carried out in the presence of an organic amide. Examples of such organic amides include cyclic and acyclic C₁ to C₆ amides such as N,N-dimethylacetamide, N-methylpyrrolidone and N,N-dimethylformamide. In the context of the present invention, N,N-dimethylformamide is preferred.

In another embodiment of the present invention, the reaction is conveniently conducted at ambient temperature or at an elevated temperature, preferably at an elevated temperature, more preferably at a temperature in the range of from about 90° C. to about 100° C., and most preferably at a temperature of about 95° C.

In another embodiment of the present invention, the reaction is carried out for an extended period of time, preferably from about 1 hour to about several days, more preferably from about 5 hours to few days, preferably about 60 hours.

In a preferred embodiment of the present invention, after the imino chloride, compound 3, is obtained, the reaction mixture is filtered and the filtrate is washed with water.

In another embodiment of the present invention, the reaction is conducted in the presence of a base. A base usable in the context of the present invention can be either an inorganic base or an organic base.

Non-limiting examples of the inorganic base can include metal hydroxides such as sodium hydroxide and potassium hydroxide; metal carbonates such as sodium carbonate and potassium carbonate; and metal hydrogen carbonates such as sodium hydrogen carbonate and potassium hydrogen carbonate.

In a preferred embodiment of the present invention, the inorganic base is selected from the group consisting of metal carbonates such as sodium carbonate and potassium carbonate, and is preferably potassium carbonate.

Non-limiting examples of the organic base, on the other hand, can include tertiary alkylamines such as triethylamine, tributylamine and N,N-diisopropylethylamine; dialkylanilines such as N,N-dimethylaniline and N,N-diethylaniline; heterocyclic amines such as pyridine, N,N-dimethylaminopyridine and N-methylmorpholine; metal alkoxides such as sodium methoxide, sodium ethoxide, sodium isopropoxide and potassium tert-butoxide; 1,8-diazobicyclo[5,4,0]undecene and N-benzyltrimethylammonium hydroxide or a combination thereof.

In yet another embodiment of the present invention, the impurities may be optionally removed by conventional techniques well known in the art. The impurities described hereinabove can be removed, if desired, by any suitable separation procedure such as, for example, filtration, extraction, column chromatography, thin-layer chromatography, preparative low, medium or high-pressure liquid chromatography or a combination of these procedures. Specific illustrations of suitable separation procedures are described in the Examples section that follows. However, other equivalent separation or isolation procedures could, of course, also be used. Filtration, extraction and a combination of these procedures are the presently most preferred separation procedures.

In yet another embodiment of the present invention, to assist in impurity removal, it is effective to treat the substrate with an adsorbent, preferably with active charcoal. Activated charcoal is added to the organic solution containing quetiapine free base. If desired, a filter-aid may be additionally added. After the activated charcoal has been added, stirring is continued at constant temperature for between 5 and 60 minutes, preferably between 10 and 30 minutes, most preferably about 15 minutes, and the mixture obtained is filtered to remove the solids.

After removing the impurities, the free base product is recovered, for example by separation from the solvent or other volatiles. In a preferred embodiment, the pressure is reduced, and the temperature is raised slightly to evaporate the solvent or other volatiles. After evaporation, the yellow-colored oily product, quetiapine free base, is obtained, substantially free of impurities. The purity of the product is preferably greater than 99%, more preferably is about 99.2% as measured by HPLC.

In another embodiment of the present invention, the pure free base product may be converted to a corresponding acid addition salt. Preferably, these salts are pharmaceutically acceptable salts.

In yet another embodiment of the present invention, the conversion is accomplished by treatment with at least a stoichiometric amount of an appropriate acid. According to the present invention, the appropriate acid includes, but is not limited to inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. A preferred salt is the hemi-fumarate salt.

Typically, the quetiapine free base prepared by the process described herein above is dissolved in an alkanol solvent such as ethanol or methanol and the like, and the acid is added to the mixture. The temperature is maintained between 0° C. and the reflux temperature, preferably the temperature is maintained between 0° C. and 25° C. The resulting crystals precipitate spontaneously or may be brought out of solution by cooling the mixture or by adding a less polar solvent.

In yet another embodiment of the present invention, the isolated crystals can be dried using conventionally known methods to give pure quetiapine hemi-fumarate. The drying stage may be carried out by increasing the temperature or reducing the pressure or a combination of both. Non-limiting examples of drying technologies or equipments usable in context of the present invention include vacuum ovens, tray ovens, rotary ovens and fluidized bed dryers.

The quetiapine hemi-fumarate is obtained by this process having a purity greater than 99%, preferably about 99.8% as measured by HPLC.

In another embodiment of the present invention, the crystalline quetiapine addition salt may be purified by converting the said salt to the corresponding free base by treatment with at least a stoichiometric equivalent of a suitable organic or inorganic base such as described hereinabove and converting the pure free base product again to a corresponding acid addition salt.

In another embodiment of the present invention, the crystalline quetiapine hemi-fumarate may be re-crystallized by any conventional re-crystallization method known in the art.

In yet another embodiment of the present invention, the processes described hereinabove for the preparation of quetiapine and the corresponding acid addition salts thereof may be conveniently and inexpensively scaled-up.

Although, the following examples illustrate the practice of the present invention in some of its embodiments, the examples should not be construed as limiting the scope of the invention. Other embodiments will be apparent to one skilled in the art from consideration of the specification and examples. It is intended that the specification, including the examples, is considered exemplary only, with the scope and spirit of the invention being indicated by the claims which follow.

### EXAMPLES

The samples of quetiapine hemi-fumarate and related substances were determined using HPLC system equipped with Inertsil ODS-P, 5 μm, 250×4.6 mm of GL Science, and a UV detector operated on 220 nm. Analyses were performed using the following mobile phase, at flow rate of 1.0 ml/minute, temperature of 30° C., and run time of 50 minutes.

Solution A: 0.01M Na₂HPO₄ buffer adjusted to pH 7.0 with concentrated H₃PO₄.

Solution B: Methanol

Solution C: Acetonitrile

Mobile phase: A mixture of 30% A, 60% B and 10% C.

The retention time of quetiapine hemi-fumarate is about 11 minutes and the retention time of the imino chloride is about 17 minutes.

### Example 1

A 250 ml three-necked round-bottom flask equipped with a mechanical stirrer, nitrogen inlet and a Dean-Stark apparatus was charged with toluene (75 ml), which was dried by azeotropic distillation. Dry DMF was added (3.7 ml, 0.048 mol) and the reaction mixture was cooled by ice-water bath. Oxalyl chloride was added drop-wise (4.24 ml, 0.048 mol) followed by adding dibenzo[b,f][1,4]thiazepine-11(10-H)-one in one portion (10 g, 0.044 mol) and the reaction mixture was heated to reflux for about 21 hours. The reaction mixture was cooled and the solvent was removed by rotary evaporator. The residue was dissolved in dichloromethane and washed with water. The layers were separated and activated charcoal was added to the organic layer containing the imino chloride. The suspension thus obtained was stirred for at least 15 minutes at elevated temperature (between 90° C. and 100° C.) and then hot-filtered. The filter was rinsed with warm dichloromethane. The filtrate was dried over magnesium sulfate and the solvent was removed by rotary evaporator to obtain 8 g of the imino chloride in 66% yield, (91% pure by HPLC) as a brown-colored solid.

### Example 2

A 1000 ml reaction vessel equipped with a magnetic stirrer and with a reflux condenser was charged with toluene (110 ml) and water (21 ml) and the solution was stirred. 1-(2-hydroxyethoxy)ethylpiperazine (27 g, 0.155 mol) and additional volume of toluene (100 ml) were added. Finally, 11-chloro-dibenzo[b,f][1,4]-thiazepine (35 g, 0.142 mol) and potassium carbonate (21 g, 0.152 mol) were added and the two-phase reaction mixture was heated to reflux. After about 20 hours the reaction mixture was cooled to 80° C. Water was added (190 ml) and the reaction mixture was cooled to 25° C. and stirred for 30 minutes. The mixture was filtered and the isolated solid was washed with toluene (35 ml). The two layers of the filtrate were separated. The organic phase was washed with water (210 ml). The combined aqueous layers were washed with toluene (105 ml). The organic layers, containing the quetiapine free base, were combined and activated charcoal was added and the suspension thus obtained was stirred for at least 15 minutes at elevated temperature (between 90° C. and 100° C.) and then hot-filtered. The filter was rinsed with warm toluene (50 ml). The filtrate and washings were combined and the toluene was distilled out at atmospheric pressure (about 260 ml). The mixture was cooled to 60° C. and ethanol was added (700 ml). Fumaric acid was added in portions (8.4 g) and the mixture was stirred for 15 minutes at 60° C. The mixture was cooled to 25° C. and stirred for 1 hour, during which time the salt began to crystallize. The mixture was cooled to 5° C. and stirred for another 1 hour. The crystallized product was collected by filtration, washed with cold ethanol and dried at 50° C. in vacuum to afford 58 g (92% yield) of quetiapine hemi-fumarate as a white solid (99.9% pure by HPLC).

### Example 3

A 250 ml reaction vessel equipped with a magnetic stirrer and a reflux condenser was charged with toluene (30 ml). 1-(2-hydroxyethoxy)ethylpiperazine (7.8 g, 44.8 mmol), potassium carbonate (5.6 g, 40.6 mmol) and 11-chloro-dibenzo[b,f][1,4]-thiazepine (10 g, 40.8 mmol) were added. The mixture was heated to reflux for 6 hours. The mixture was cooled to 25° C. and washed twice with water (total volume: 90 ml). The organic layers were combined and activated charcoal was added to the combined organic layers containing the quetiapine free base. The suspension thus obtained was stirred for at least 15 minutes at elevated temperature (between 90° C. and 100° C.) and then hot-filtered. The filter was rinsed with warm toluene (50 ml). The filtrate and washings were combined, dried over magnesium sulfate and the solvent was removed by rotary evaporator to yield a yellow-colored oil. The oil was dissolved in ethanol (70 ml) and fumaric acid was added (2.4 g, 20.7 mmol) and the mixture was stirred for 30 minutes at 25° C. during which time the salt began to crystallize. The crystallized product was collected by filtration, washed with cold ethanol and dried at 50° C. in vacuum to afford 16.1 g (89.5% yield) of quetiapine hemi-fumarate as a white solid (99.9% pure by HPLC, m.p. −171-172° C).

### Example 4

A 250 ml reaction vessel equipped with a magnetic stirrer and a reflux condenser was charged with toluene (20 ml), 1-(2-hydroxyethoxy)ethylpiperazine (3.5 g, 20 mmol), 11-chloro-dibenzo[b,f][1,4]-thiazepine (5 g, 20 mmol) and triethylamine (7.7 g, 76 mmol). The reaction mixture was heated to 95° C. for about 60 hours. After work-up, the free base was obtained as a brown oil. The product was identified by TLC analysis, using methanol:dichloromethane (1:9) as eluent. The oil was dissolved in ethanol (30 ml) and fumaric acid was added (0.55 g, 5 mmol) and the mixture was stirred for 30 minutes at 25° C. during which time the salt began to crystallize. The crystallized product was collected by filtration, washed with cold ethanol and dried at 50° C. in vacuum to afford 3.4 g (about 40% yield) of quetiapine hemi-fumarate as a white solid (99% pure by HPLC).

### Example 5

A 250 ml three-necked round-bottom flask equipped with a mechanical stirrer, nitrogen inlet and a Dean Stark apparatus was charged with toluene (100 ml), which was dried by azeotropic distillation. Dry DMF was added (3.7 ml, 0.048 mol) and the reaction mixture was cooled using an ice-water bath. Oxalyl chloride was added drop-wise (4.24 ml, 0.048 mol). Dibenzo[b,f][1,4]thiazepine-11(10-H)-one was added in one portion (10 g, 0.044 mol) and the reaction mixture was heated to 95° C. for about 17 hours. The reaction mixture was filtered and the solid was washed with toluene (50 ml). The filtrate was washed twice with water (150 ml) and concentrated to three quarters of the initial volume by using rotary evaporator. The concentrated reaction mixture was dried by azeotropic distillation and 1-(2-hydroxyethoxy)ethylpiperazine (6.2 g, 0.036 mol) and potassium carbonate (5.4 g, 0.039 mol) were added and the reaction mixture was heated to reflux. After about 43 hours the reaction mixture was cooled to room temperature and washed twice with water (130 ml) and once with HCl 1N (50 ml). The layers were separated and the acidic extract was treated with 20% NaOH solution (30 ml) and washed with dichloromethane (50 ml). The organic layers were combined and activated charcoal was added to the combined organic layers containing the quetiapine free base. The suspension thus obtained was stirred for at least 15 minutes at elevated temperature (between 90° C. and 100° C.) and then hot-filtered. The filter was rinsed with warm toluene (50 ml). The filtrate was dried over magnesium sulfate and the solvent was removed by rotary evaporator to yield 9 g (49% yield, 99.2% pure by HPLC) of quetiapine free base as a yellow-colored oil. The oil was dissolved in ethanol (15 ml) and fumaric acid was added in portions (1.4 g, 0.012 mol). The mixture was stirred for 1 hour, during which time the salt began to crystallize. The mixture was cooled to 5° C. and stirred for another 1 hour. The crystallized product was collected by filtration, washed with cold ethanol and dried at 50° C. in vacuum to afford 8.6 g (44% yield) of quetiapine hemi-fumarate as a white solid (99.8% pure by HPLC).

What is claimed is:

1. A two-step process for preparing quetiapine and the corresponding acid addition salts thereof comprising the steps of:
a) Reacting dibenzo[b,f][1,4]thiazepine-11(10-H)-one, compound 2, with halogenating agent in an organic solvent to yield compound 3; and
b) Reacting the product resulting from step a) with 1-(2-hydroxyethoxy)ethylpiperazine, compound 4, or its salt, in the presence of a base.

2. The process for obtaining quetiapine and the corresponding acid addition salts thereof, according to claim 1, wherein said step a) comprises:
a) Charging the reaction vessel with an organic solvent and optionally drying the solvent by azeotropic distillation;
b) Adding an amide and optionally cooling the reaction mixture;
c) Adding a chlorinating agent drop-wise followed by adding compound 2;
d) Refluxing the reaction mixture for sufficient time to allow completing the reaction; and
e) Working-up the reaction mixture thus obtaining compound 3.

3. The process according to claim 2, wherein said drying of the organic solvent is by continuous removal of water using, for example, a Dean-Stark trap or activated molecular sieves.

4. The process according to claim 2, wherein the organic solvent is selected from the group consisting of ethylbenzene, toluene, xylenes, and the like and mixtures thereof.

5. The process according to claim 4, wherein the organic solvent is toluene.

6. The process according to claim 2, wherein the organic amide is selected from the group consisting of N,N-dimethylacetamide, N-methylpyrrolidone, and N,N-dimethylformamide.

7. The process according to claim 6, wherein the amide is N,N-dimethylformamide.

8. The process according to claim 2, wherein the chlorinating agent is selected from the group consisting of thionyl chloride, phosphorous pentachloride, phosphorous oxychloride, oxalyl chloride, N-chlorosuccinimide or N-chlorobenzotriazole.

9. The process according to claim 8, wherein the chlorinating agent is oxalyl chloride.

10. The process for obtaining quetiapine and the corresponding acid addition salts thereof, according to claim 1, wherein said second step b) comprises:
a) Reacting 11-chloro-dibenzo[b,f][1,4]-thiazepine, compound 3, with 1-(2-hydroxyethoxy)ethylpiperazine or its salt, compound 3, in the presence of a base in an organic solvent or in a two-phase solvent system;
b) Filtering the reaction mixture, washing and optionally treating the organic phase with activated charcoal; and
c) Isolating the pure product as a corresponding acid addition salt thereof.

11. The process according to claim 10, wherein the organic solvent is selected from the group consisting of ethylbenzene, toluene, xylenes, and the like and mixtures thereof.

12. The process according to claim 10, wherein the base is an organic base or an inorganic base.

13. The process according to claim 12, wherein said organic base is selected from the group consisting of tertiary alkylamines such as triethylamine, tributylamine and N,N-diisopropylethylamine; dialkylanilines such as N,N-dimethylaniline and N,N-diethylaniline; heterocyclic amines such as pyridine, substituted pyridines, N,N-dimethylaminopyridine and N-methylmorpholine; metal alkoxides such as sodium methoxide, sodium ethoxide, sodium isopropoxide and potassium tert-butoxide; 1,8-diazobicyclo[5,4,0]undecene and N-benzyltrimethylammonium hydroxide, and a combination thereof.

14. The process according to claim 13, wherein the base is triethylamine.

15. The process according to claim 12, wherein said inorganic base is selected from the group consisting of metal hydroxides, such as sodium hydroxide and potassium hydroxide, metal carbonates such as sodium carbonate and potassium carbonate, and metal hydrogen carbonates such as sodium hydrogen carbonate and potassium hydrogen carbonate, and combinations thereof.

16. The process according to claim 15, wherein the base is potassium carbonate.

17. The process according to claim 10, wherein the said two-phase solvent system is prepared by combining a water-immiscible organic solvent and an aqueous phase.

18. The process according to claim 17, wherein said water-immiscible organic solvent is selected from the group consisting of ethylbenzene, toluene, xylenes and the like and a mixture thereof.

19. The process according to claim 17, wherein the said aqueous phase comprising an inorganic base.

20. The process according to claim 19, wherein said inorganic base is selected from the group consisting of metal hydroxides, such as sodium hydroxide and potassium hydroxide, metal carbonates such as sodium carbonate and potassium carbonate, and metal hydrogen carbonates such as sodium hydrogen carbonate and potassium hydrogen carbonate, and combinations thereof.

21. The process according to claim 20, wherein the inorganic base is potassium carbonate.

22. The process according to claim 10, wherein said acid is selected from the group consisting of inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

23. The process according to claim 22, wherein the acid is fumaric acid, thus pharmaceutically pure quetiapine hemi-fumarate is prepared, having purity greater than 99%, preferably having a purity equal to or greater than 99.8%.

24. A one-pot process for preparing quetiapine and the corresponding acid addition salts thereof, the process comprising reacting dibenzo[b,f][1,4]thiazepine-11(10-H)-one, compound 2, with halogenating agent in an organic solvent to yield compound 3 in situ and reacting the thus obtained compound 3 with 1-(2-hydroxyethoxy)ethylpiperazine, compound 4, or its salt, in the presence of a base.

25. The process according to claim 24, wherein the organic solvent is dried and maintained water free during the reaction by continues removal of water using, for example, a Dean-Stark trap or activated molecular sieves.

26. The process according to claim 24, wherein the organic solvent is selected from the group consisting of ethylbenzene, toluene, xylenes, and the like, and mixtures thereof.

27. The process according to claim 26, wherein the organic solvent is toluene.

28. The process according to claim 24 further comprises adding an organic amide selected from the group consisting of N,N-dimethylacetamide, N-methylpyrrolidone and N,N-dimethylformamide.

29. The process according to claim 24, wherein the halogenating agent is preferably a chlorinating agent selected from the group consisting of thionyl chloride, phosphorous pentachloride, phosphorous oxychloride, oxalyl chloride, N-chlorosuccinimide or N-chlorobenzotriazole.

30. The process according to claim 29, wherein the chlorinating agent is oxalyl chloride.

31. The process according to claim 24, wherein the base is an inorganic base or organic base.

32. The process according to claim 31, wherein the inorganic base is selected from the group consisting of metal hydroxides such as sodium hydroxide and potassium hydroxide, metal carbonates such as sodium carbonate and potassium carbonate, and metal hydrogen carbonates such as sodium hydrogen carbonate and potassium hydrogen carbonate, and combinations thereof.

33. The process according to claim 32, wherein the base is potassium carbonate.

34. The process according to claim 24, wherein said organic base is selected from the group consisting of tertiary alkylamines such as triethylamine, tributylamine and N,N-diisopropylethylamine, dialkylanilines such as N,N-dimethylaniline and N,N-diethylaniline, heterocyclic amines such as pyridine, N,N-dimethylaminopyridine and N-methylmorpholine, metal alkoxides such as sodium methoxide, sodium ethoxide, sodium isopropoxide and potassium tert-butoxide, 1,8-diazobicyclo[5,4,0]undecene, N-benzyltrimethylammonium hydroxide, and a combination thereof.

35. The process according to claim 24, wherein the said acid is selected from the group consisting of inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

36. The process according to claim 35, wherein the acid is fumaric acid, thus pharmaceutically pure quetiapine hemi-fumarate is prepared, having purity greater than 99%, preferable having a purity equal to or greater than 99.8%.
